(19) United States
(12) Patent Application Publication
(10) Pub. No.: US 2002/0082539 A1
(43) Pub. Date: Jun. 27, 2002

(54) Reinforced, adhesive wound closure and method of manufacturing

(75) Inventors: Debra J. Battah, Lake Elmo, MN (US); Terry W. Lewis, Woodbury, MN (US); John E. Riedel, Hugo, MN (US)

Correspondence Address: Attention: Robert Sprague, Office of Intellectual Property Counsel, 3M Innovative Properties Company, P.O. Box 33427, St. Paul, MN, 55133-3427, US

(73) Assignee: 3M Innovative Properties Company

(21) Appl. No.: 09/747,601

(22) Filed: Dec. 22, 2000

(51) Int. Cl. A61F 13/00

(52) U.S. Cl. 602/43; 602/41

(57) ABSTRACT

Adhesive wound closure devices and methods of manufacturing the devices and the material for the devices are disclosed. The devices, methods, and materials address the requirements for tensile strength and skin adhesion by microcreping adhesive wound closure material that includes a nonwoven fabric web with one or more reinforcing fibers bonded thereto.

### TECHNICAL FIELD

The invention relates to adhesive wound closure devices including reinforcing fibers, methods of making the devices, and methods of manufacturing adhesive wound closure material.

### BACKGROUND

In medicine, sutures have long been used to close serious wounds. More recently, adhesive closures have been introduced which can effectively close some types of wounds without needing to inflict the additional injury inherent in suturing. These adhesive closures have a backing and a layer of adhesive suitable for adhering to the skin. Because this kind of wound closure needs to resist sometimes substantial forces tending to reopen a wound, closures having backings with long fibers to reinforce them in the longitudinal, or cross-wound, direction have been particularly successful. For example, STERI-STRIPS wound closures, commercially available from Minnesota Mining and Manufacturing Company (St. Paul, Minn.), are constructed in this way.

Although such wound closures need to have good tensile strength, they also need good adhesion to the skin on each side of the wound to be successful. The use of the reinforcing fibers provide the first, but they tend to interfere with the second; i.e., the reinforcing fibers impart stiffness which makes a strong adhesive bond to the skin more difficult to achieve.

### SUMMARY OF THE INVENTION

The present invention addresses the requirements for tensile strength and skin adhesion by microcreping adhesive wound closure material that includes a nonwoven fabric web with one or more reinforcing fibers bonded thereto. The resulting microcreped adhesive wound closure material provides a desirable combination of tensile strength (along the direction of the reinforcing fiber or fibers) and conformability to the skin.

The bond between the non-woven fabric and the reinforcing fibers in the backing material is relatively weak and the strong compacting forces and resultant heat generated during microcreping may be expected to destroy that bond. Contrary to expectations, however, the reinforcing fibers react by buckling and remain sufficiently bonded to the non-woven fabric after microcreping.

At the other end of the spectrum, it could also be expected that the bond between the non-woven fabric and the reinforcing fibers could be significantly strengthened due to the compacting forces and heat experienced during microcreping. Surprisingly, that is also not the result, with the bond strength between the non-woven fabric and the reinforcing fibers remaining low enough such that the reinforced web retains sufficient extensibility to improve skin adhesion after an adhesive is applied thereto. That improved adhesion is observed without a corresponding loss in the longitudinal tensile strength of the web.

After microcreping, the length of the web (or the adhesive wound closure backing) will typically be shorter than its original length, where the original length is measured before microcreping. It may be preferred that the length of the web (or the adhesive wound closure backing) be no more than 99.5% of its original length. At the other end of the spectrum, it may be preferred that the length of the web after microcreping (or the adhesive wound closure backing) is at least 85% of its original length. One preferred level of microcreping may result in a web (or an adhesive wound closure backing) that has post-microcreped length of 95% of its original length. The lengths discussed herein are measured while the web is not under tension.

In one aspect, the present invention provides a method of making adhesive wound closure material by providing a web of non-woven fabric including binder fibers; bonding at least one longitudinal reinforcing fiber to the web of non-woven fabric, wherein the at least one reinforcing fiber is coextensive with a length of the web; microcreping the non-woven fabric and the at least one reinforcing fiber so as to compress the non-woven fabric and the at least one reinforcing fiber along the length of the web; and applying a layer of pressure sensitive adhesive to the web.

In another aspect, the present invention provides method of making adhesive wound closure devices by providing a web that includes a non-woven fabric having binder fibers; bonding at least one longitudinal reinforcing fiber to the non-woven fabric, wherein the at least one reinforcing fiber is coextensive with a length of the web; microcreping the non-woven fabric and the at least one reinforcing fiber so as to compress the non-woven fabric and the at least one reinforcing fiber along a length of the web; applying a layer of pressure sensitive adhesive to the web to form an adhesive-coated microcreped web including the non-woven fabric and the at least one reinforcing fiber; and converting the adhesive-coated microcreped web into a plurality of adhesive wound closure devices.

In another aspect the present invention provides an adhesive wound closure device having a backing including a non-woven backing with binder fibers; at least one longitudinal reinforcing fiber bonded to the non-woven fabric, wherein the at least one reinforcing fiber is coextensive with a length of the backing; and wherein the non-woven fabric and the at least one reinforcing fiber are microcreped so as to compress the non-woven fabric and the at least one reinforcing fiber along the length of the backing. The device also includes a layer of pressure sensitive adhesive on the backing.

These and other features and advantages of the present invention are described below with respect to various illustrative embodiments of the invention.

### DEFINITIONS

“Microcreping,” as used herein, means mechanically compacting a fabric so that it has a smaller lengthwise dimension than it originally had. For example, “microcreped fabric” is a fabric that has been mechanically compacted so that it has a smaller lengthwise dimension than it had before microcreping.

### BRIEF DESCRIPTION OF THE DRAWING

In the several figures of the attached drawing, like parts bear like reference numerals, and:

FIG. 1 illustrates a perspective view of a wound closure made according to the present invention;

FIG. 2 illustrates an end cross-section view taken along section lines 2-2 in FIG. 1;

FIGS. 3, 4, and 5 illustrate variations of one microcreping process that may be used in connection with the present invention; and

FIG. 6 illustrates another microcreping process that may be used in connection with the present invention.

### DETAILED DESCRIPTION OF ILLUSTRATIVE EMBODIMENTS OF THE INVENTION

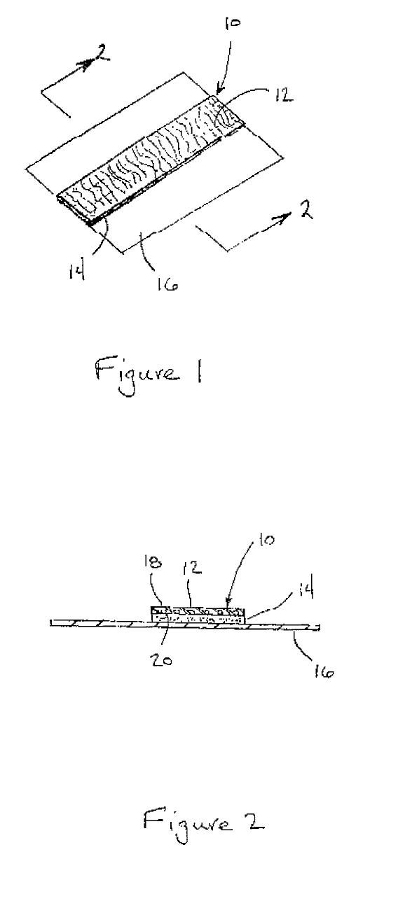

Referring now to FIG. 1, an exemplary wound closure 10 according to the present invention is illustrated. The wound closure 10 has a microcreped reinforced web 12 having a layer of adhesive 14 applied thereto. Conveniently, the wound closure 10 may be supplied on a release liner 16 for ease of handling before use.

Referring now to FIG. 2, a cross section view taken along section lines 2-2 in FIG. 1 is illustrated. In this view it can be more readily appreciated that the microcreped reinforced web 12 includes a nonwoven fabric 18 and at least one, preferably a plurality, of reinforcing fibers 20. Each of the reinforcing fibers 20 preferably has a length that is coextensive with the length of the nonwoven fabric 18.

The nonwoven fabric 18 must include some portion of binder fibers. The binder fibers have at least an outer portion that is bondable, i.e., meltable or fusable, when subjected to thermal energy such as may be provided by heat, sonic, or laser energy sources. The temperature at which at least the outer portion of the binder fibers melt is preferably in the range of 225° F. to 400° F. (110° C. to 200° C.), more preferably 225° F. to 250° F. (110° C. to 125° C.). The binder fibers can be of any polymeric fiber-forming material such as polyester, polyamide, polyolefin, or combinations thereof provided that at least the outer portion is thermally bondable. Preferred binder fibers are of the core-and-sheath-type, sometimes called “melty” fibers, having a sheath which melts to form bonds at the desired temperature and a core which melts at a temperature at least 50° F. (30° C.) above the melting temperature of the sheath.

The binder fibers preferably have a denier in the range of 0.5 to 10 denier, more preferably 1 to 6 denier. The finer denier fibers generally provide greater softness to the sheet material. The binder fibers may be of a combination of different lengths and/or deniers to achieve a desired balance of properties. Two denier polyester staple fibers, commercially available as Celbond type K54, from Kanematsu, and Type 254 fibers commercially available from Hoechst Celanese of Charlotte, N.C. may be used.

The nonwoven fabric 18 may preferably include some fibers that do not thermobond, or at least do not thermobond at the temperatures that activate the binder fibers. Such non-binding fibers include, e.g., polymeric staple fibers such as polyester, nylon, polypropylene, acetate, rayon, and acrylic, as well as natural fibers, such as cotton, and wood pulp. The addition of fibrous material to the nonwoven fabric 18 may generally increase the softness and suppleness of the sheet material. Rayon staple fibers of 1.5 DPF, commercially available as Type 8645, from Lenzing Fibers Corp., may be suitable fibrous material. Various ratios of binder fibers to non-binding fibers may be used in connection with the present invention, but ratios of approximately 1:3 of binder fibers to non-binding fibers may be preferred.

The reinforcing fibers 20 are preferably capable of being thermally adhered to the nonwoven fabric 18 by partial melting of the binder fibers through the action of heat and/or pressure, and are conveniently continuous multifilament thermoplastic yarns. A 100 denier partially oriented polyester yarn, with 33 ends/yam, commercially available as #612 from Unific is considered suitable. Two yarns from Hoechst Celanese, namely #680 which is a 100 denier polyester yarn with 36 ends/yarn, and #610 which is a 150 denier polyester yarn with 33 ends/yarn, are also considered suitable.

While it is possible to form the nonwoven fabric 18 completely separately and then thermally bond the reinforcing fibers 20 to the formed nonwoven fabric 18 in a second operation to form the reinforced web, it may be preferred to accomplish this in a unified operation. To do this, a mixture of binder and non-binding fibers is laid on a conveyor using a fiber processing machine. For example a Hergeth model, once sold commercially by Hergeth-Hollingsworth is considered suitable. The conveyor carries this mixture to a heated nip roll; a roll heated to about 160° C. is considered suitable. The reinforcing fibers are also brought to the nip, preferably with a separation between reinforcing fibers being accomplished by, e.g., a comb upstream of the nip. A density of approximately 8 strands of reinforcing fibers/cm in the cross-web direction is considered suitable.

Although the processes described above bond the reinforcing fibers to the base web to form an integrated backing for the wound closure, the additional step of applying a binding agent may be preferred to develop the required degree of strength in the integrated backing. Latex binding agents adapted to be applied in liquid form may be particularly suitable. Included among suitable binders are Hystretch V43 from BF Goodrich of Cleveland, Ohio; binder 78-6283 from National Starch of Bridgewater, N.J.; and binders 1019 and St-954 from Rohm & Haas of Philadelphia, Pa. Conveniently, the integrated backing is brought to a dipping station with the selected liquid binding agent, the agent having been diluted and pH adjusted as needed. It may be convenient to include an antifoaming agent in this dip, and Antifoam B, commercially available from Dow Corning Corp. of Midland, Mich. is considered to be suitable. A squeegee arrangement may be used to remove excess binding agent from the web, and the reinforced web is then dried and wound.

The reinforced web is then ready for microcreping to impart stretchability and conformability to the web. Two commercial microcreping processes are believed to be capable of treating fabrics of the present invention, although other processes that provide the desired microcreping may be used. One such process is commercialized by the Micrex Corporation of Walpole, Mass. (the “Micrex” process). A second such process is commercialized by the Tubular Textile Machinery Corporation of Lexington, N.C. (the “TTM” process). The TTM process is similar in principle to the Micrex process, although certain details are different. In the TTM process, the web is passed into the compacting zone over a feed roll and under a shoe. The web is then compacted or microcreped by contacting a lower compacting shoe and a retarding roll. Nevertheless, in both processes the web is subjected to a compaction force along its length due to frictional retarders.

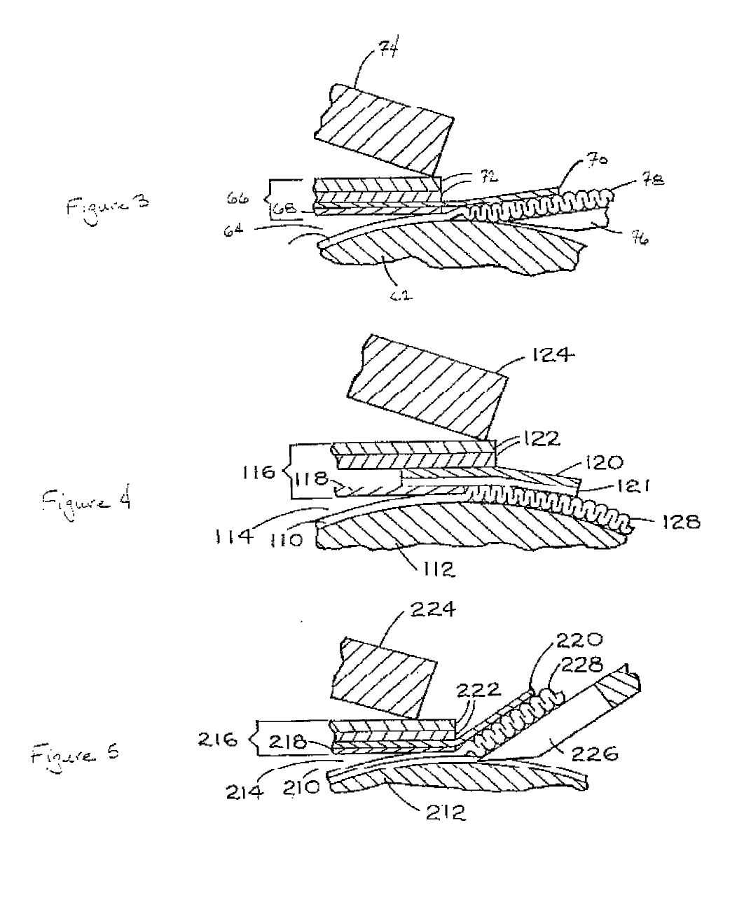

As depicted in FIG. 3, a reinforced web 12, supported by a main roll 62, is introduced into a converging passage 64 between the main roll and a movable retarder 66. The movable retarder has a primary surface 68, a flexible retarder 70, and one or more back-up blades 72. The movable retarder is held against the reinforced web using a pressure plate 74. The web passes through a secondary passage between the flexible retarder and a rigid retarder 76 and exits as a microcreped reinforced web 78.

As depicted in FIG. 4, a reinforced web 110, supported by a main roll 112, is introduced into a converging passage 114 between the main roll and a movable retarder 116. The movable retarder has a primary surface 118, a flexible retarder 120, a frictional retarder 121, and one or more back-up blades 122. The movable retarder is held against the reinforced web using a pressure plate 124. The web passes through a secondary passage between the frictional retarder 121 and the main roll 112 and exits as a microcreped reinforced web 128.

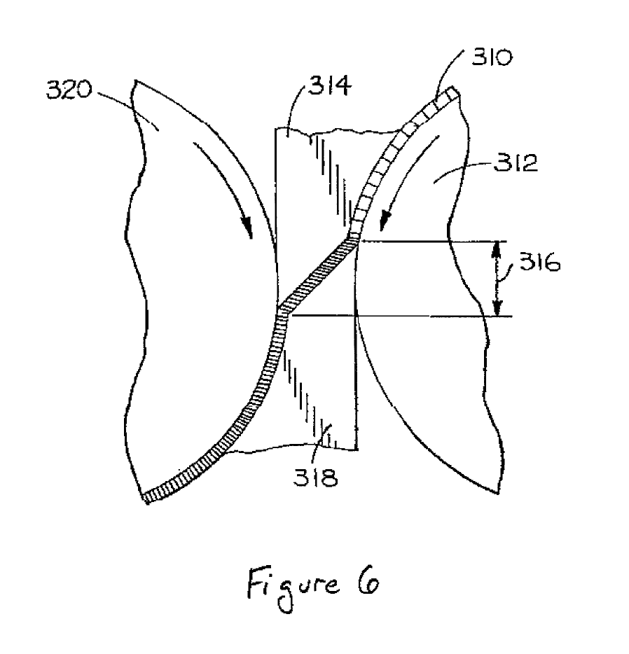

As depicted in FIG. 5, a reinforced web 210, supported by a main roll 212, is introduced into a converging passage 214 between the main roll and a movable retarder 216. The movable retarder has a primary surface 218, a flexible retarder 220, and one or more back-up blades 222. The movable retarder is held against the reinforced web using a pressure plate 224. The web passes through a secondary passage between the flexible retarder and a comb 226 and exits as a microcreped reinforced 228.

As depicted in FIG. 6, a reinforced web 310, supported by a feed roll 312, is introduced into a passage between an upper compacting shoe 314 and the feed roll. The web then passes into a compacting zone 316 between the upper compacting shoe and a lower compacting shoe 318. The web becomes compacted in this zone and exits through a passage between the lower compacting shoe and a retarding roll 320.

The microcreped, reinforced web is then ready to be coated with a skin compatible adhesive layer. Preferred pressure sensitive adhesives which can be used in the adhesive layer of the present invention are the normal adhesives which are applied to the skin, such as the acrylate copolymers described in U.S. Pat. No. RE 24,906, e.g., a 97:3 weight ratio iso-octyl acrylate:acrylamide copolymer or a 96:4 weight ratio iso-octyl acrylate:acrylamide copolymer. Other medical grade skin adhesives such as copolymers of iso-octyl acrylate and N-vinyl pyrrolidone, or copolymers of iso-octyl acrylate and acrylic acid, can also be used. A layer of about twenty-five grams of skin compatible pressure sensitive adhesive per square meter of backing is considered suitable. It may be particularly convenient to apply the skin-compatible adhesive to the microcreped reinforcing web by laminating the web to a layer of adhesive that has already been coated on a release liner 16, which release liner will then remain with the wound closure until removed by the user just before use.

Liners which are suitable for use in the adhesive composites of the present invention can be made of kraft papers, polyethylene, polypropylene, polyester or composites of any of these materials. The liners are preferably coated with release agents such as fluorochemicals or silicones. For example, U.S. Pat. No. 4,472,480 describes low surface energy perfluorochemical liners. The preferred liners are papers, polyolefm films, or polyester films coated with silicone release materials. Examples of commercially available silicone coated release papers are POLYSLIK silicone release papers available from James River Co., H. P. Smith Division (Bedford Park, Ill.) and silicone release papers supplied by Daubert Chemical Co. (Dixon, Ill.). Presently considered particularly suitable as a liner is 1-60BKG-157 paper liner available from Daubert, which is a super calendered Kraft paper with a water-based silicone release surface.

Other combinations of adhesives and liners are contemplated for use with embodiments according to the present invention. Those skilled in the art will be familiar with the processes of testing a new adhesive against different liners or a new liner against different adhesives to arrive at the combination of qualities desired in a final product. Some considerations pertinent to the selection of a silicone release liner can be found in Chapter 18 of the Handbook of Pressure Sensitive Adhesive Technology, Van Nostrand-Reinhold, 1982, pp. 384-403. U.S. Pat. No. 4,472,480 also describes considerations pertinent to the selection of a perfluoropolyether release liner.

After the microcreped reinforced web includes a layer of skin-compatible pressure sensitive adhesive, the adhesive wound closure material of the present invention may be complete and ready for converting into the adhesive wound closure devices of the present invention. For example, the adhesive wound closure material may be converted into adhesive wound closure devices using conventional slitting and die cutting techniques.

Typically, the adhesive wound closure material may be manufactured as a long, indefinite length web that is converted into the individual adhesive wound closure devices. For example, the nonwoven fabric 18 of FIGS. 1 and 2 may be manufactured as a web of indefinite length with the reinforcing fibers 20 being coextensive with the nonwoven fabric 18 in the machine direction (i.e., the direction of indefinite length).

The preceding specific embodiments are illustrative of the practice of the invention. This invention may be suitably practiced in the absence of any element or item not specifically described in this document. The complete disclosures of all patents, patent applications, and publications are incorporated into this document by reference as if individually incorporated in total.

Various modifications and alterations of this invention will become apparent to those skilled in the art without departing from the scope of this invention, and it should be understood that this invention is not to be unduly limited to illustrative embodiments set forth herein, but is to be controlled by the limitations set forth in the claims and any equivalents to those limitations.

What is claimed is:

1. A method of making adhesive wound closure material, the method comprising:
providing a web that comprises a non-woven fabric comprising binder fibers;
bonding at least one longitudinal reinforcing fiber to the non-woven fabric, wherein the at least one reinforcing fiber is coextensive with a length of the web;
microcreping the non-woven fabric and the at least one reinforcing fiber so as to compress the non-woven fabric and the at least one reinforcing fiber along the length of the web; and
applying a layer of pressure sensitive adhesive to the web.

2. The method according to claim 1, wherein the length of the web after microcreping is shorter than its length before microcreping.

3. The method according to claim 1, wherein the length of the web after microcreping is no more than 99.5% of its length before microcreping.

4. The method according to claim 1, wherein the length of the web after microcreping is at least 85% of its length before microcreping.

5. The method according to claim 1, wherein bonding of the at least one reinforcing fiber comprises thermal bonding between at least some of the binder fibers and the at least one reinforcing fiber.

6. The method according to claim 1, wherein the non-woven fabric comprises non-binding fibers, and wherein the at least one reinforcing fiber is not bonded to the non-binding fibers.

7. The method according to claim 1, further comprising applying a binding agent to the web and the at least one reinforcing fiber before microcreping.

8. The method according to claim 1, wherein applying the pressure sensitive adhesive comprises applying the pressure sensitive adhesive on the at least one reinforcing fiber and the non-woven fabric.

9. A method of making adhesive wound closure devices, the method comprising:
providing a web that comprises a non-woven fabric comprising binder fibers;
bonding at least one longitudinal reinforcing fiber to the non-woven fabric, wherein the at least one reinforcing fiber is coextensive with a length of the web;
microcreping the non-woven fabric and the at least one reinforcing fiber so as to compress the non-woven fabric and the at least one reinforcing fiber along a length of the web;
applying a layer of pressure sensitive adhesive to the web to form an adhesive-coated microcreped web comprising the non-woven fabric and the at least one reinforcing fiber; and
converting the adhesive-coated microcreped web into a plurality of adhesive wound closure devices.

10. The method according to claim 9, wherein the length of the web after microcreping is shorter than its length before microcreping.

11. The method according to claim 9, wherein the length of the web after microcreping is no more than 99.5% of its length before microcreping.

12. The method according to claim 9, wherein the length of the web after microcreping is at least 85% of its length before microcreping.

13. The method according to claim 9, wherein bonding of the at least one reinforcing fiber comprises thermal bonding between at least some of the binder fibers and the at least one reinforcing fiber.

14. The method according to claim 9, wherein the non-woven fabric comprises non-binding fibers, and wherein the at least one reinforcing fiber is not bonded to the non-binding fibers.

15. The method according to claim 9, further comprising applying a binding agent to the web and the at least one reinforcing fiber before microcreping.

16. The method according to claim 9, wherein applying the pressure sensitive adhesive comprises applying the pressure sensitive adhesive on the at least one reinforcing fiber and the non-woven fabric.

17. An adhesive wound closure device comprising:
a backing comprising:
non-woven fabric comprising binder fibers;
at least one longitudinal reinforcing fiber bonded to the non-woven fabric, wherein the at least one reinforcing fiber is coextensive with a length of the backing;
wherein the non-woven fabric and the at least one reinforcing fiber are microcreped so as to compress the non-woven fabric and the at least one reinforcing fiber along the length of the backing;
a layer of pressure sensitive adhesive on the backing.

18. The device of claim 17, wherein the length of the microcreped backing is shorter than its length before microcreping.

19. The device of claim 17, wherein the length of the microcreped backing is no more than 99.5% of its length before microcreping.

20. The device of claim 17, wherein the length of the microcreped backing is at least 85% of its length before microcreping.

21. The device of claim 17, wherein the at least one reinforcing fiber is thermally bonded to at least some of the binder fibers.

22. The device of claim 17, wherein the backing comprises non-binding fibers, and wherein the at least one reinforcing fiber is not bonded to the non-binding fibers.
